(19) United States
(12) Patent Application Publication
(10) Pub. No.: US 2020/0050837 A1
(43) Pub. Date: Feb. 13, 2020

(54) SYSTEM AND METHOD FOR DETECTING INVISIBLE HUMAN EMOTION

(71) Applicant: NURALOGIX CORPORATION, Toronto (CA)

(72) Inventors: Kang Lee, Toronto (CA); Pu Zheng, Toronto (CA)

(21) Appl. No.: 16/592,939

(22) Filed: Oct. 4, 2019

Related U.S. Application Data
(63) Continuation of application No. 14/868,601, filed on Sep. 29, 2015.
(60) Provisional application No. 62/058,227, filed on Oct. 1, 2014.

(51) Int. Cl. G06K 9/00; G16H 50/20; G06K 9/66; G06T 5/50; G09B 19/00; G06K 9/62

(52) U.S. Cl. CPC G06K 9/00281; G16H 50/20; G06K 9/00315; G06K 9/66; G06T 5/50; G06K 2209/05; G06K 9/6278; G06T 2207/10016; G06T 2207/20224; G06K 2009/00939; G09B 19/00

(57) ABSTRACT

A system and method for emotion detection and more specifically to an image-capture based system and method for detecting invisible and genuine emotions felt by an individual. The system provides a remote and non-invasive approach by which to detect invisible emotion with a high confidence. The system enables monitoring of hemoglobin concentration changes by optical imaging and related detection systems.

### TECHNICAL FIELD

The following relates generally to emotion detection and more specifically to an image-capture based system and method for detecting invisible human emotion.

### BACKGROUND

Humans have rich emotional lives. More than 90% of the time, we experience rich emotions internally but our facial expressions remain neutral. These invisible emotions motivate most of our behavioral decisions. How to accurately reveal invisible emotions has been the focus of intense scientific research for over a century. Existing methods remain highly technical and/or expensive, making them only accessible for heavily funded medical and research purposes, but are not available for wide everyday usage including practical applications, such as for product testing or market analytics.

Non-invasive and inexpensive technologies for emotion detection, such as computer vision, rely exclusively on facial expression, thus are ineffective on expressionless individuals who nonetheless experience intense internal emotions that are invisible. Extensive evidence exists to suggest that physiological signals such as cerebral and surface blood flow can provide reliable information about an individual's internal emotional states, and that different emotions are characterized by unique patterns of physiological responses. Unlike facial-expression-based methods, physiological-information-based methods can detect an individual's inner emotional states even when the individual is expressionless. Typically, researchers detect such physiological signals by attaching sensors to the face or body. Polygraphs, electromyography (EMG) and electroencephalogram (EEG) are examples of such technologies, and are highly technical, invasive, and/or expensive. They are also subjective to motion artifacts and manipulations by the subject.

Several methods exist for detecting invisible emotion based on various imaging techniques. While functional magnetic resonance imaging (fMRI) does not require attaching sensors to the body, it is prohibitively expensive and susceptible to motion artifacts that can lead to unreliable readings. Alternatively, hyperspectral imaging may be employed to capture increases or decreases in cardiac output or “blood flow” which may then be correlated to emotional states. The disadvantages present with the use of hyperspectral images include cost and complexity in terms of storage and processing.

### SUMMARY

In one aspect, a system for detecting invisible human emotion expressed by a subject from a captured image sequence of the subject is provided, the system comprising an image processing unit trained to determine a set of bitplanes of a plurality of images in the captured image sequence that represent the hemoglobin concentration (HC) changes of the subject, and to detect the subject's invisible emotional states based on HC changes, the image processing unit being trained using a training set comprising a set of subjects for which emotional state is known.

In another aspect, a method for detecting invisible human emotion expressed by a subject is provided, the method comprising: capturing an image sequence of the subject, determining a set of bitplanes of a plurality of images in the captured image sequence that represent the hemoglobin concentration (HC) changes of the subject, and detecting the subject's invisible emotional states based on HC changes using a model trained using a training set comprising a set of subjects for which emotional state is known.

A method for invisible emotion detection is further provided.

### BRIEF DESCRIPTION OF THE DRAWINGS

The features of the invention will become more apparent in the following detailed description in which reference is made to the appended drawings wherein:

FIG. 1 is an block diagram of a transdermal optical imaging system for invisible emotion detection;

FIG. 2 illustrates re-emission of light from skin epidermal and subdermal layers;

FIG. 3 is a set of surface and corresponding transdermal images illustrating change in hemoglobin concentration associated with invisible emotion for a particular human subject at a particular point in time;

FIG. 4 is a plot illustrating hemoglobin concentration changes for the forehead of a subject who experiences positive, negative, and neutral emotional states as a function of time (seconds).

FIG. 5 is a plot illustrating hemoglobin concentration changes for the nose of a subject who experiences positive, negative, and neutral emotional states as a function of time (seconds).

FIG. 6 is a plot illustrating hemoglobin concentration changes for the cheek of a subject who experiences positive, negative, and neutral emotional states as a function of time (seconds).

FIG. 7 is a flowchart illustrating a fully automated transdermal optical imaging and invisible emotion detection system;

FIG. 8 is an exemplary report produced by the system;

FIG. 9 is an illustration of a data-driven machine learning system for optimized hemoglobin image composition;

FIG. 10 is an illustration of a data-driven machine learning system for multidimensional invisible emotion model building;

FIG. 11 is an illustration of an automated invisible emotion detection system; and

FIG. 12 is a memory cell.

### DETAILED DESCRIPTION

Embodiments will now be described with reference to the figures. For simplicity and clarity of illustration, where considered appropriate, reference numerals may be repeated among the Figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

Various terms used throughout the present description may be read and understood as follows, unless the context indicates otherwise: “or” as used throughout is inclusive, as though written “and/or”; singular articles and pronouns as used throughout include their plural forms, and vice versa; similarly, gendered pronouns include their counterpart pronouns so that pronouns should not be understood as limiting anything described herein to use, implementation, performance, etc. by a single gender; “exemplary” should be understood as “illustrative” or “exemplifying” and not necessarily as “preferred” over other embodiments. Further definitions for terms may be set out herein; these may apply to prior and subsequent instances of those terms, as will be understood from a reading of the present description.

Any module, unit, component, server, computer, terminal, engine or device exemplified herein that executes instructions may include or otherwise have access to computer readable media such as storage media, computer storage media, or data storage devices (removable and/or non-removable) such as, for example, magnetic disks, optical disks, or tape. Computer storage media may include volatile and non-volatile, removable and non-removable media implemented in any method or technology for storage of information, such as computer readable instructions, data structures, program modules, or other data. Examples of computer storage media include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by an application, module, or both. Any such computer storage media may be part of the device or accessible or connectable thereto. Further, unless the context clearly indicates otherwise, any processor or controller set out herein may be implemented as a singular processor or as a plurality of processors. The plurality of processors may be arrayed or distributed, and any processing function referred to herein may be carried out by one or by a plurality of processors, even though a single processor may be exemplified. Any method, application or module herein described may be implemented using computer readable/executable instructions that may be stored or otherwise held by such computer readable media and executed by the one or more processors.

The following relates generally to emotion detection and more specifically to an image-capture based system and method for detecting invisible human emotional, and specifically the invisible emotional state of an individual captured in a series of images or a video. The system provides a remote and non-invasive approach by which to detect an invisible emotional state with a high confidence.

The sympathetic and parasympathetic nervous systems are responsive to emotion. It has been found that an individual's blood flow is controlled by the sympathetic and parasympathetic nervous system, which is beyond the conscious control of the vast majority of individuals. Thus, an individual's internally experienced emotion can be readily detected by monitoring their blood flow. Internal emotion systems prepare humans to cope with different situations in the environment by adjusting the activations of the autonomic nervous system (ANS); the sympathetic and parasympathetic nervous systems play different roles in emotion regulation with the former regulating up fight-flight reactions whereas the latter serves to regulate down the stress reactions. Basic emotions have distinct ANS signatures. Blood flow in most parts of the face such as eyelids, cheeks and chin is predominantly controlled by the sympathetic vasodilator neurons, whereas blood flowing in the nose and ears is mainly controlled by the sympathetic vasoconstrictor neurons; in contrast, the blood flow in the forehead region is innervated by both sympathetic and parasympathetic vasodilators. Thus, different internal emotional states have differential spatial and temporal activation patterns on the different parts of the face. By obtaining hemoglobin data from the system, facial hemoglobin concentration (HC) changes in various specific facial areas may be extracted. These multidimensional and dynamic arrays of data from an individual are then compared to computational models based on normative data to be discussed in more detail below. From such comparisons, reliable statistically based inferences about an individual's internal emotional states may be made. Because facial hemoglobin activities controlled by the ANS are not readily subject to conscious controls, such activities provide an excellent window into an individual's genuine innermost emotions.

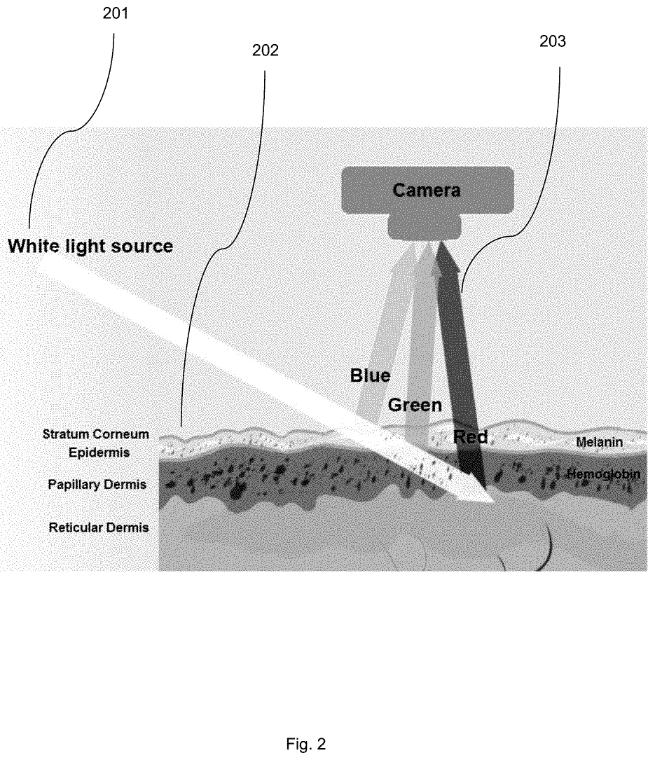

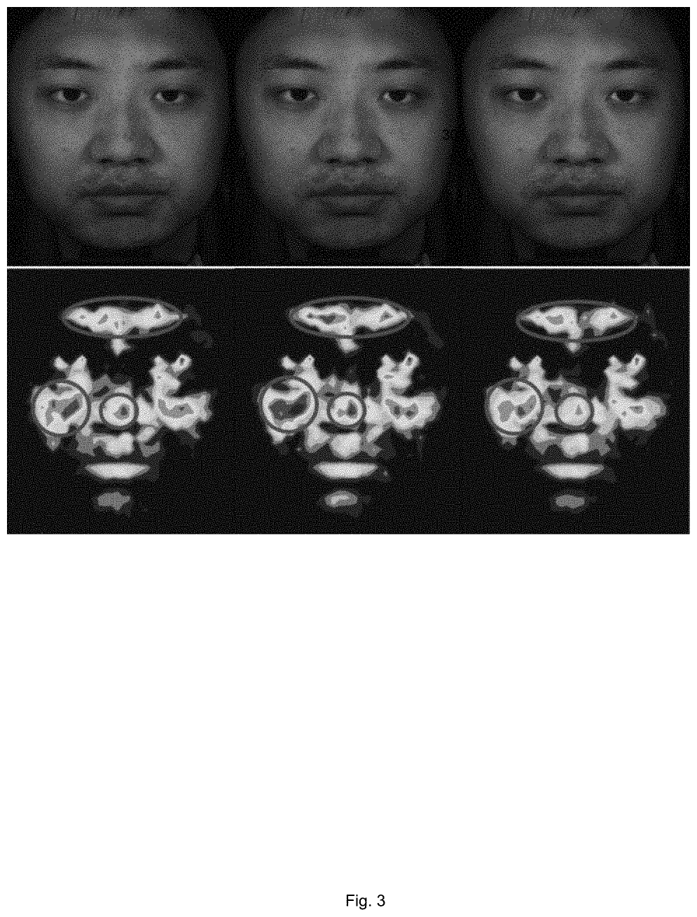

It has been found that it is possible to isolate hemoglobin concentration (HC) from raw images taken from a traditional digital camera, and to correlate spatial-temporal changes in HC to human emotion. Referring now to FIG. 2, a diagram illustrating the re-emission of light from skin is shown. Light (201) travels beneath the skin (202), and re-emits (203) after travelling through different skin tissues. The re-emitted light (203) may then be captured by optical cameras. The dominant chromophores affecting the re-emitted light are melanin and hemoglobin. Since melanin and hemoglobin have different color signatures, it has been found that it is possible to obtain images mainly reflecting HC under the epidermis as shown in FIG. 3.

The system implements a two-step method to generate rules suitable to output an estimated statistical probability that a human subject's emotional state belongs to one of a plurality of emotions, and a normalized intensity measure of such emotional state given a video sequence of any subject. The emotions detectable by the system correspond to those for which the system is trained.

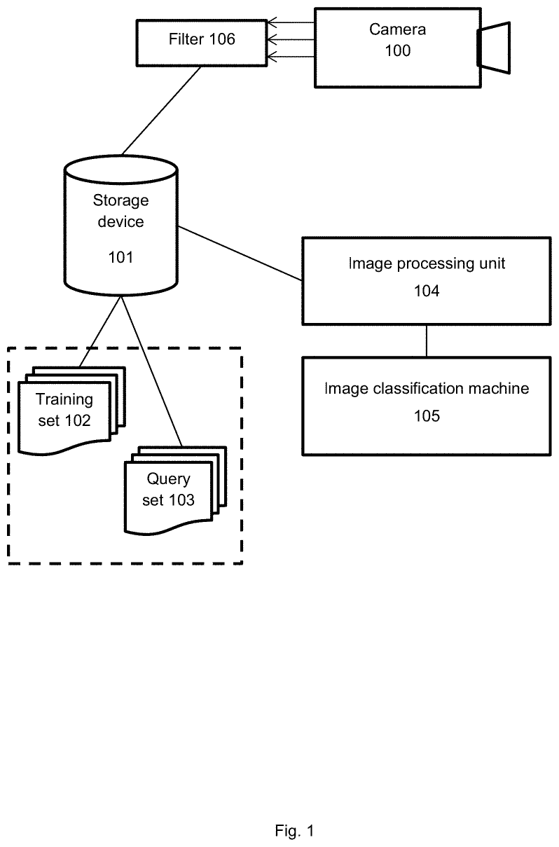

Referring now to FIG. 1, a system for invisible emotion detection is shown. The system comprises interconnected elements including an image processing unit (104), an image filter (106), and an image classification machine (105). The system may further comprise a camera (100) and a storage device (101), or may be communicatively linked to the storage device (101) which is preloaded and/or periodically loaded with video imaging data obtained from one or more cameras (100). The image classification machine (105) is trained using a training set of images (102) and is operable to perform classification for a query set of images (103) which are generated from images captured by the camera (100), processed by the image filter (106), and stored on the storage device (102).

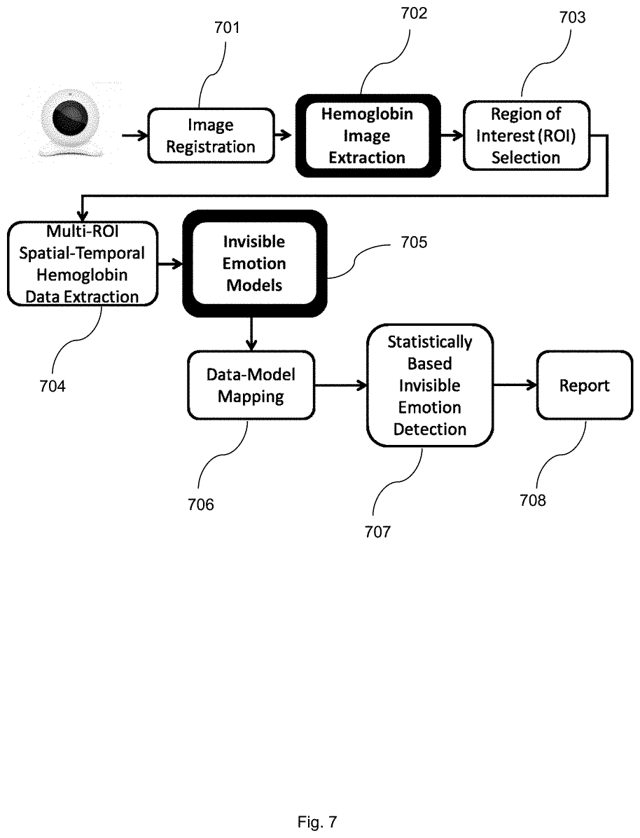

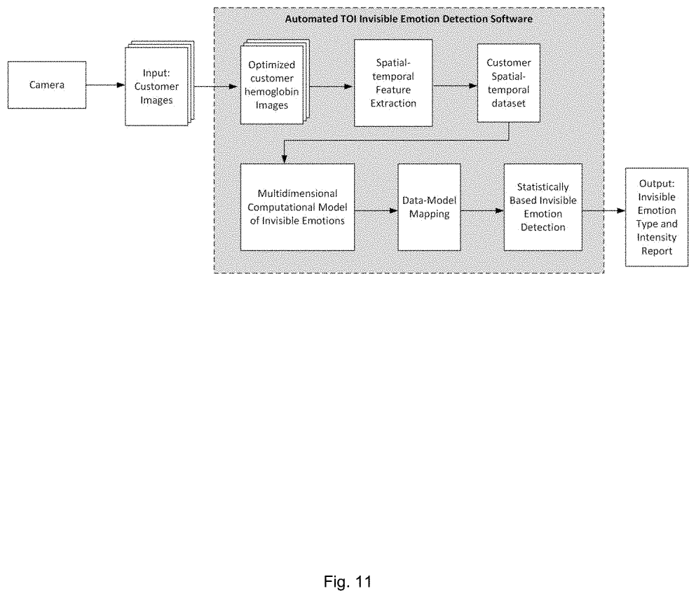

Referring now to FIG. 7, a flowchart illustrating a fully automated transdermal optical imaging and invisible emotion detection system is shown. The system performs image registration 701 to register the input of a video sequence captured of a subject with an unknown emotional state, hemoglobin image extraction 702, ROI selection 703, multi-ROI spatial-temporal hemoglobin data extraction 704, invisible emotion model 705 application, data mapping 706 for mapping the hemoglobin patterns of change, emotion detection 707, and report generation 708. FIG. 11 depicts another such illustration of automated invisible emotion detection system.

The image processing unit obtains each captured image or video stream and performs operations upon the image to generate a corresponding optimized HC image of the subject. The image processing unit isolates HC in the captured video sequence. In an exemplary embodiment, the images of the subject's faces are taken at 30 frames per second using a digital camera. It will be appreciated that this process may be performed with alternative digital cameras and lighting conditions.

Isolating HC is accomplished by analyzing bitplanes in the video sequence to determine and isolate a set of the bitplanes that provide high signal to noise ratio (SNR) and, therefore, optimize signal differentiation between different emotional states on the facial epidermis (or any part of the human epidermis). The determination of high SNR bitplanes is made with reference to a first training set of images constituting the captured video sequence, coupled with EKG, pneumatic respiration, blood pressure, laser Doppler data from the human subjects from which the training set is obtained. The EKG and pneumatic respiration data are used to remove cardiac, respiratory, and blood pressure data in the HC data to prevent such activities from masking the more-subtle emotion-related signals in the HC data. The second step comprises training a machine to build a computational model for a particular emotion using spatial-temporal signal patterns of epidermal HC changes in regions of interest (“ROIs”) extracted from the optimized “bitplaned” images of a large sample of human subjects.

For training, video images of test subjects exposed to stimuli known to elicit specific emotional responses are captured. Responses may be grouped broadly (neutral, positive, negative) or more specifically (distressed, happy, anxious, sad, frustrated, intrigued, joy, disgust, angry, surprised, contempt, etc.). In further embodiments, levels within each emotional state may be captured. Preferably, subjects are instructed not to express any emotions on the face so that the emotional reactions measured are invisible emotions and isolated to changes in HC. To ensure subjects do not “leak” emotions in facial expressions, the surface image sequences may be analyzed with a facial emotional expression detection program. EKG, pneumatic respiratory, blood pressure, and laser Doppler data may further be collected using an EKG machine, a pneumatic respiration machine, a continuous blood pressure machine, and a laser Doppler machine and provides additional information to reduce noise from the bitplane analysis, as follows.

ROIs for emotional detection (e.g., forehead, nose, and cheeks) are defined manually or automatically for the video images. These ROIs are preferably selected on the basis of knowledge in the art in respect of ROIs for which HC is particularly indicative of emotional state. Using the native images that consist of all bitplanes of all three R, G, B channels, signals that change over a particular time period (e.g., 10 seconds) on each of the ROIs in a particular emotional state (e.g., positive) are extracted. The process may be repeated with other emotional states (e.g., negative or neutral). The EKG and pneumatic respiration data may be used to filter out the cardiac, respirator, and blood pressure signals on the image sequences to prevent non-emotional systemic HC signals from masking true emotion-related HC signals. Fast Fourier transformation (FFT) may be used on the EKG, respiration, and blood pressure data to obtain the peek frequencies of EKG, respiration, and blood pressure, and then notch filers may be used to remove HC activities on the ROIs with temporal frequencies centering around these frequencies. Independent component analysis (ICA) may be used to accomplish the same goal.

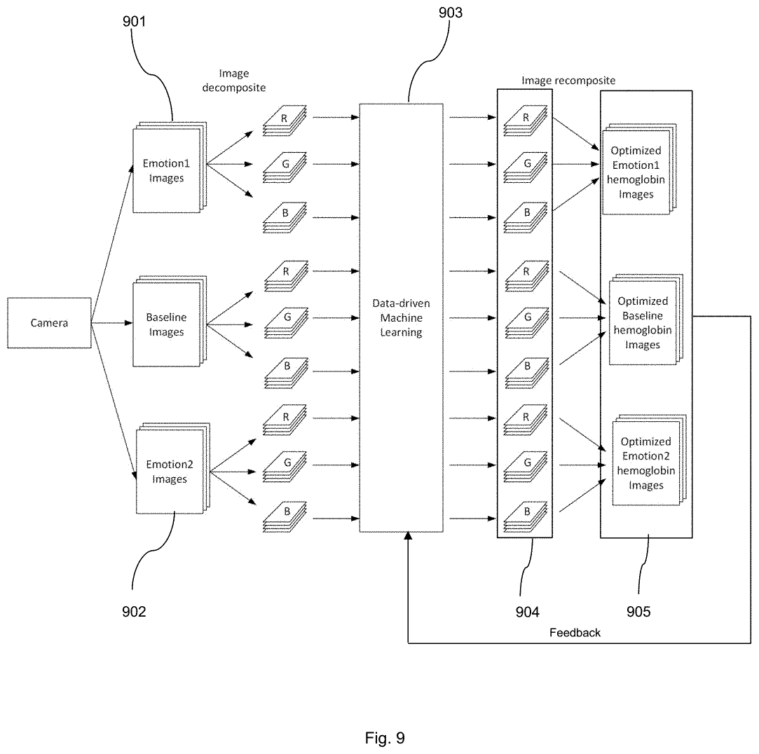

Referring now to FIG. 9 an illustration of data-driven machine learning for optimized hemoglobin image composition is shown. Using the filtered signals from the ROIs of two or more than two emotional states 901 and 902, machine learning 903 is employed to systematically identify bitplanes 904 that will significantly increase the signal differentiation between the different emotional state and bitplanes that will contribute nothing or decrease the signal differentiation between different emotional states. After discarding the latter, the remaining bitplane images 905 that optimally differentiate the emotional states of interest are obtained. To further improve SNR, the result can be fed back to the machine learning 903 process repeatedly until the SNR reaches an optimal asymptote.

The machine learning process involves manipulating the bitplane vectors (e.g., 8×8×8, 16×16×16) using image subtraction and addition to maximize the signal differences in all ROIs between different emotional states over the time period for a portion (e.g., 70%, 80%, 90%) of the subject data and validate on the remaining subject data. The addition or subtraction is performed in a pixel-wise manner. An existing machine learning algorithm, the Long Short Term Memory (LSTM) neural network, GPNet, or a suitable alternative thereto is used to efficiently and obtain information about the improvement of differentiation between emotional states in terms of accuracy, which bitplane(s) contributes the best information, and which does not in terms of feature selection. The Long Short Term Memory (LSTM) neural network and GPNet allow us to perform group feature selections and classifications. The LSTM and GPNet machine learning algorithm are discussed in more detail below. From this process, the set of bitplanes to be isolated from image sequences to reflect temporal changes in HC is obtained. An image filter is configured to isolate the identified bitplanes in subsequent steps described below.

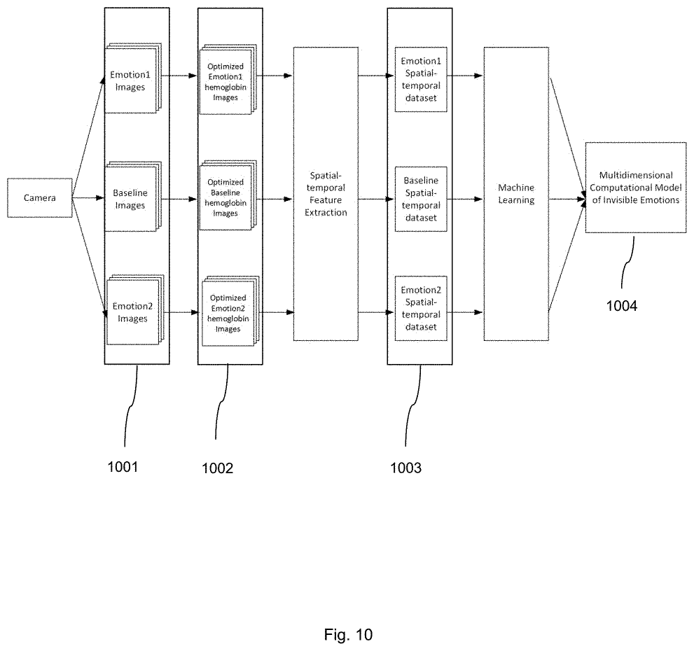

The image classification machine 105, which has been previously trained with a training set of images captured using the above approach, classifies the captured image as corresponding to an emotional state. In the second step, using a new training set of subject emotional data derived from the optimized biplane images provided above, machine learning is employed again to build computational models for emotional states of interests (e.g., positive, negative, and neural). Referring now to FIG. 10, an illustration of data-driven machine learning for multidimensional invisible emotion model building is shown. To create such models, a second set of training subjects (preferably, a new multi-ethnic group of training subjects with different skin types) is recruited, and image sequences 1001 are obtained when they are exposed to stimuli eliciting known emotional response (e.g., positive, negative, neutral). An exemplary set of stimuli is the International Affective Picture System, which has been commonly used to induce emotions and other well established emotion-evoking paradigms. The image filter is applied to the image sequences 1001 to generate high HC SNR image sequences. The stimuli could further comprise non-visual aspects, such as auditory, taste, smell, touch or other sensory stimuli, or combinations thereof.

Using this new training set of subject emotional data 1003 derived from the bitplane filtered images 1002, machine learning is used again to build computational models for emotional states of interests (e.g., positive, negative, and neural) 1003. Note that the emotional state of interest used to identify remaining bitplane filtered images that optimally differentiate the emotional states of interest and the state used to build computational models for emotional states of interests must be the same. For different emotional states of interests, the former must be repeated before the latter commences.

The machine learning process again involves a portion of the subject data (e.g., 70%, 80%, 90% of the subject data) and uses the remaining subject data to validate the model. This second machine learning process thus produces separate multidimensional (spatial and temporal) computational models of trained emotions 1004.

To build different emotional models, facial HC change data on each pixel of each subject's face image is extracted (from Step 1) as a function of time when the subject is viewing a particular emotion-evoking stimulus. To increase SNR, the subject's face is divided into a plurality of ROIs according to their differential underlying ANS regulatory mechanisms mentioned above, and the data in each ROI is averaged.

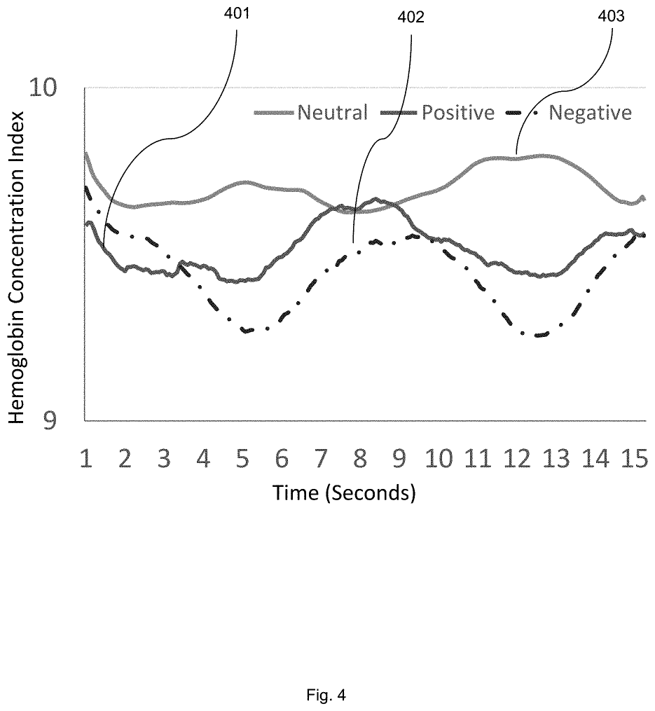

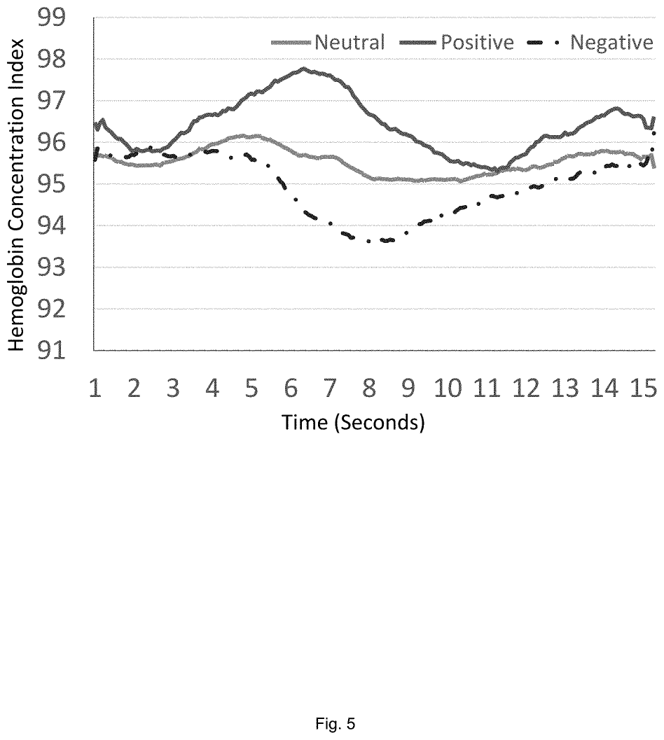

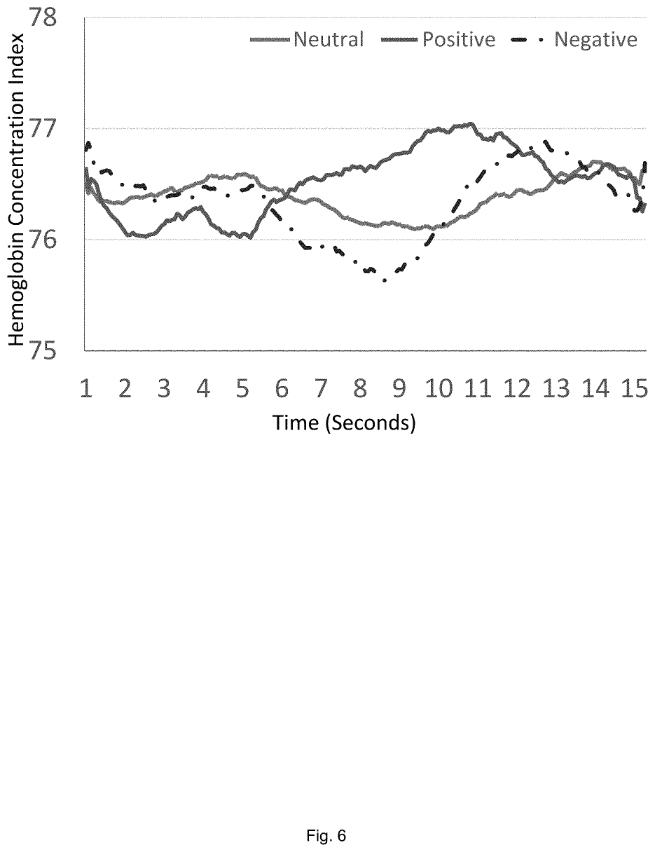

Referring now to FIG. 4, a plot illustrating differences in hemoglobin distribution for the forehead of a subject is shown. Though neither human nor computer-based facial expression detection system may detect any facial expression differences, transdermal images show a marked difference in hemoglobin distribution between positive 401, negative 402 and neutral 403 conditions. Differences in hemoglobin distribution for the nose and cheek of a subject may be seen in FIG. 5 and FIG. 6 respectively.

The Long Short Term Memory (LSTM) neural network, GPNet, or a suitable alternative such as non-linear Support Vector Machine, and deep learning may again be used to assess the existence of common spatial-temporal patterns of hemoglobin changes across subjects. The Long Short Term Memory (LSTM) neural network or GPNet machine or an alternative is trained on the transdermal data from a portion of the subjects (e.g., 70%, 80%, 90%) to obtain a multi-dimensional computational model for each of the three invisible emotional categories. The models are then tested on the data from the remaining training subjects.

Following these steps, it is now possible to obtain a video sequence of any subject and apply the HC extracted from the selected biplanes to the computational models for emotional states of interest. The output will be (1) an estimated statistical probability that the subject's emotional state belongs to one of the trained emotions, and (2) a normalized intensity measure of such emotional state. For long running video streams when emotional states change and intensity fluctuates, changes of the probability estimation and intensity scores over time relying on HC data based on a moving time window (e.g., 10 seconds) may be reported. It will be appreciated that the confidence level of categorization may be less than 100%.

In further embodiments, optical sensors pointing, or directly attached to the skin of any body parts such as for example the wrist or forehead, in the form of a wrist watch, wrist band, hand band, clothing, footwear, glasses or steering wheel may be used. From these body areas, the system may also extract dynamic hemoglobin changes associated with emotions while removing heart beat artifacts and other artifacts such as motion and thermal interferences.

In still further embodiments, the system may be installed in robots and their variables (e.g., androids, humanoids) that interact with humans to enable the robots to detect hemoglobin changes on the face or other-body parts of humans whom the robots are interacting with. Thus, the robots equipped with transdermal optical imaging capacities read the humans' invisible emotions and other hemoglobin change related activities to enhance machine-human interaction.

Two example implementations for (1) obtaining information about the improvement of differentiation between emotional states in terms of accuracy, (2) identifying which bitplane contributes the best information and which does not in terms of feature selection, and (3) assessing the existence of common spatial-temporal patterns of hemoglobin changes across subjects will now be described in more detail. The first such implementation is a recurrent neural network and the second is a GPNet machine.

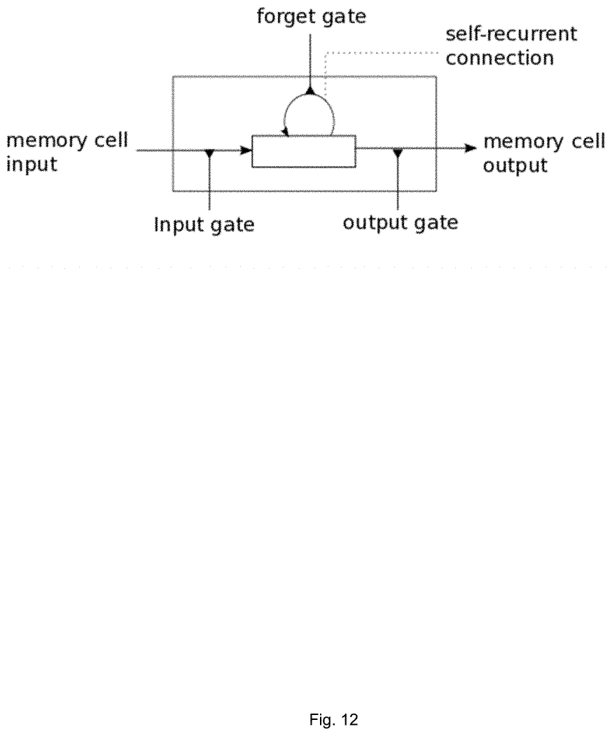

One recurrent neural network is known as the Long Short Term Memory (LSTM) neural network, which is a category of neural network model specified for sequential data analysis and prediction. The LSTM neural network comprises at least three layers of cells. The first layer is an input layer, which accepts the input data. The second (and perhaps additional) layer is a hidden layer, which is composed of memory cells (see FIG. 12). The final layer is output layer, which generates the output value based on the hidden layer using Logistic Regression.

Each memory cell, as illustrated, comprises four main elements: an input gate, a neuron with a self-recurrent connection (a connection to itself), a forget gate and an output gate. The self-recurrent connection has a weight of 1.0 and ensures that, barring any outside interference, the state of a memory cell can remain constant from one time step to another. The gates serve to modulate the interactions between the memory cell itself and its environment. The input gate permits or prevents an incoming signal to alter the state of the memory cell. On the other hand, the output gate can permit or prevent the state of the memory cell to have an effect on other neurons. Finally, the forget gate can modulate the memory cell's self-recurrent connection, permitting the cell to remember or forget its previous state, as needed.

The equations below describe how a layer of memory cells is updated at every time step t. In these equations:

xₜ is the input array to the memory cell layer at time t. In our application, this is the blood flow signal at all ROIs

\vec{x}ₜ=_{└}x₁ₜx₂ₜ [symbol]
Wᵢ, W_{f}, W_{c}, Wₒ, Uᵢ, U_{f}, U_{c}, Uₒ and Vₒ are weight matrices; and
   bᵢ, b_{f}, b_{c} an bₒ are bias vectors

First, we compute the values for iₜ, the input gate, and [symbol] the candidate value for the states of the memory cells at time t:

iₜ=σ(Wᵢxₜ+Uᵢhₜ₋₁+bᵢ)

$$\text{?} ( {W}_{c} {x}_{t} + {U}_{c} {h}_{t - 1} + {b}_{c} )$$

$$\text{?} \text{indicates text missing or illegible when filed}$$

Second, we compute the value for fₜ, the activation of the memory cells' forget gates at time t:

fₜ=σ(W_{f}xₜ+U_{f}hₜ₋₁+b_{f})

Given the value of the input gate activation iₜ, the forget gate activation fₜ and the candidate state value [symbol] , we can compute Cₜ the memory cells' new state at time t:

$${C}_{t} = {i}_{t} * \text{?} \text{?} \text{?}$$

$$\text{?} \text{indicates text missing or illegible when filed}$$

With the new state of the memory cells, we can compute the value of their output gates and, subsequently, their outputs:

oₜ=σ(Wₒxₜ+Uₒhₜ₋₁+VₒCₜ+bₒ)

hₜ=oₜ*tan h(Cₜ)

Based on the model of memory cells, for the blood flow distribution at each time step, we can calculate the output from memory cells. Thus, from an input sequence x₀, x₁, x₂, . . . - -, the memory cells in the LSTM layer will produce a representation sequence h₀, h₁, h₂, . . . - -.

The goal is to classify the sequence into different conditions. The Logistic Regression output layer generates the probability of each condition based on the representation sequence from the LSTM hidden layer. The vector of the probabilities at time step t can be calculated by:

pₜ=softmax(Wₒᵤₜₚᵤₜhₜ+bₒᵤₜₚᵤₜ)

where Wₒᵤₜₚᵤₜ is the weight matrix from the hidden layer to the output layer, and bₒᵤₜₚᵤₜ is the bias vector of the output layer. The condition with the maximum accumulated probability will be the predicted condition of this sequence.

The GPNet computational analysis comprises three steps (1) feature extraction, (2) Bayesian sparse-group feature selection and (3) Bayesian sparse-group feature classification.

For each subject, using surface images, transdermal images or both, concatenated feature vectors ν_{T1}, ν_{T2}, ν_{T3}, ν_{T4} may be extracted for conditions T2, T3, and T4 etc. (e.g., baseline, positive, negative, and neutral or). Images are treated from T1 as background information to be subtracted from images of T2, T3, and T4. As an example, when classifying T2 vs T3, the difference vectors ν_{T2\1}=ν_{T2}−ν_{T1} and ν_{T3\1}=ν_{T3}−ν_{T1} are computed. Collecting the difference vectors from all subjects, two difference matrices V_{T2\1} and V_{T3\1} are formed, where each row of V_{T2\1} or V_{T3\1} is a difference vector from one subject. The matrix

$${V}_{T 2 , 3 \backslash 1} = [ \begin{matrix}{V}_{T 2 \backslash 1} \\ {V}_{T 3 \backslash 1}\end{matrix} ]$$

is normalized so that each column of it has standard deviation 1. Then the normalized V_{T2,3\1} is treated as the design matrix for the following Bayesian analysis. When classifying T4 vs T3, the same procedure of forming difference vectors and matrices, and jointly normalizing the columns of V_{T4\1} and V_{T3\1} is applied.

An empirical Bayesian approach to classify the normalized videos and jointly identify regions that are relevant for the classification tasks at various time points has been developed. A sparse Bayesian model that enables selection of the relevant regions and conversion to an equivalent Gaussian process model to greatly reduce the computational cost is provided. A probit model as the likelihood function to represent the probability of the binary states (e.g., positive vs. negative), may be used: y=[y₁, . . . , y_{N}]. Given the noisy feature vectors: X=[x₁, . . . , x_{N}], and the classifier w: p(y|X, w)=Πᵢ₌₁^{N}ϕ(yᵢw^{T}xᵢ). Where the function ϕ(⋅) is the Gaussian cumulative density function. To model the uncertainty in the classifier w, a Gaussian prior is assigned over it: p(w)=Πⱼ₌₁^{J} [symbol] (wⱼ|0,αⱼI).

Where wj are the classifier weights corresponding to an ROI at a particular time indexed by j, alpha_j controls the relevance of the j-th region, and J is the total number of the AOIs at all the time points. Because the prior has zero mean, if the variance alpha_j is very small, the weights for the j-th region will be centered around 0, indicating the j-th region has little relevance for the classification task. By contrast, if alpha_j is large, the j-th region is then important for the classification task. To see this relationship from another perspective, the likelihood function and the prior may be reparamatized via a simple linear transformation:

$$p ( y \vert X , w ) = {\prod }_{i = 1}^{N} φ ( {y}_{i} {\sum }_{j = 1}^{J} \sqrt{{α}_{j}} {w}_{y}^{T} {x}_{ij} )$$

$$p ( w ) = 𝒩 ( w \vert 0 , I )$$

Where xij is the feature vector extracted from the j-th region of the i-th subject. This model is equivalent to the previous one in the sense they give the same model marginal likelihood after integrating out the classifier w: p(y|X,α)=∫p(y|X,w)p(w|α)dα.

In this new equivalent model, alpha_j scales the classifier weight w_j. Clearly, the bigger the alpha_j, the more relevant the j-th region for classification.

To discover the relevance of each region, an empirical Bayesian strategy is adopted. The model marginal likelihood is maximized—p(y|X,alpha)—over the variance parameters, α=[α₁, . . . , α_{J}]. Because this marginal likelihood is a probabilistic distribution (i.e., it is always normalized to one), maximizing it will naturally push the posterior distribution to be concentrated in a subspace of alpha; in other words, many elements of alpha_j will have small values or even become zeros—thus the corresponding regions become irrelevant and only a few important regions will be selected.

A direct optimization of the marginal likelihood, however, would require the posterior distribution of the classifier w to be computed. Due to the high dimensionality of the data, classical Monte Carlo methods, such as Markov Chain Monte Carlo, will incur a prohibitively high computational cost before their convergence. If the posterior distribution is approximated by a Gaussian using the classical Laplace's method, which would necessitate inverting the extremely large covariance matrix of w inside some optimization iterations, the overall computational cost will be O(k d\hat{ }3) where d is the dimensionality of x and k is the number of optimization iterations. Again, the computational cost is too high.

To address this computational challenge, a new efficient sparse Bayesian learning algorithm is developed. The core idea is to construct an equivalent Gaussian process model and efficiently train the GP model, not the original model, from data. The expectation propagation is then applied to train the GP model. Its computation cost is on the order of O(N\hat{ }3), where N is the number of the subjects. Thus the computational cost is significantly reduced. After obtaining the posterior process of the GP model, an expectation maximization algorithm is then used to iteratively optimize the variance parameters alpha.

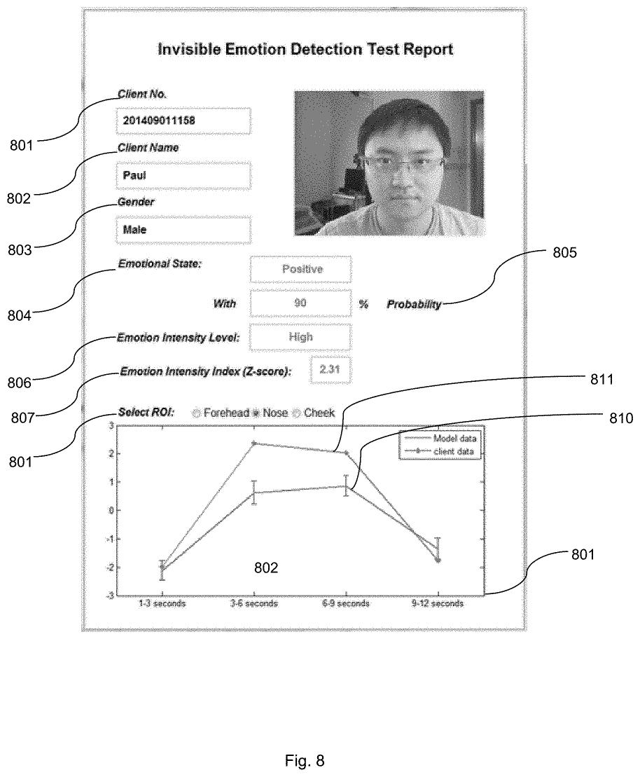

Referring now to FIG. 8, an exemplary report illustrating the output of the system for detecting human emotion is shown. The system may attribute a unique client number 801 to a given subject's first name 802 and gender 803. An emotional state 804 is identified with a given probability 805. The emotion intensity level 806 is identified, as well as an emotion intensity index score 807. In an embodiment, the report may include a graph comparing the emotion shown as being felt by the subject 808 based on a given ROI 809 as compared to model data 810, over time 811.

The foregoing system and method may be applied to a plurality of fields, including marketing, advertising and sales in particular, as positive emotions are generally associated with purchasing behavior and brand loyalty, whereas negative emotions are the opposite. In an embodiment, the system may collect videos of individuals while being exposed to a commercial advertisement, using a given product or browsing in a retail environment. The video may then be analyzed in real time to provide live user feedback on a plurality of aspects of the product or advertisement. Said technology may assist in identifying the emotions required to induce a purchase decision as well as whether a product is positively or negatively received.

In embodiments, the system may be used in the health care industry. Medical doctors, dentists, psychologist, psychiatrists, etc., may use the system to understand the real emotions felt by patients to enable better treatment, prescription, etc.

Homeland security as well as local police currently use cameras as part of customs screening or interrogation processes. The system may be used to identify individuals who form a threat to security or are being deceitful. In further embodiments, the system may be used to aid the interrogation of suspects or information gathering with respect to witnesses.

Educators may also make use of the system to identify the real emotions of students felt with respect to topics, ideas, teaching methods, etc.

The system may have further application by corporations and human resource departments. Corporations may use the system to monitor the stress and emotions of employees. Further, the system may be used to identify emotions felt by individuals interview settings or other human resource processes.

The system may be used to identify emotion, stress and fatigue levels felt by employees in a transport or military setting. For example, a fatigued driver, pilot, captain, soldier, etc., may be identified as too fatigued to effectively continue with shiftwork. In addition to safety improvements that may be enacted by the transport industries, analytics informing scheduling may be derived.

In another aspect, the system may be used for dating applicants. By understanding the emotions felt in response to a potential partner, the screening process used to present a given user with potential partners may be made more efficient.

In yet another aspect, the system may be used by financial institutions looking to reduce risk with respect to trading practices or lending. The system may provide insight into the emotion or stress levels felt by traders, providing checks and balances for risky trading.

The system may be used by telemarketers attempting to assess user reactions to specific words, phrases, sales tactics, etc. that may inform the best sales method to inspire brand loyalty or complete a sale.

In still further embodiments, the system may be used as a tool in affective neuroscience. For example, the system may be coupled with a MRI or NIRS or EEG system to measure not only the neural activities associated with subjects' emotions but also the transdermal blood flow changes. Collected blood flow data may be used either to provide additional and validating information about subjects' emotional state or to separate physiological signals generated by the cortical central nervous system and those generated by the autonomic nervous system. For example, the blush and brain problem in fNIRS (functional near infrared spectroscopy) research where the cortical hemoglobin changes are often mixed with the scalp hemoglobin changes may be solved.

In still further embodiments, the system may detect invisible emotions that are elicited by sound in addition to vision, such as music, crying, etc. Invisible emotions that are elicited by other senses including smell, scent, taste as well as vestibular sensations may also be detected.

It will be appreciated that while the present application described a system and method for invisible emotion detection, the system and method could alternatively be applied to detection of any other condition for which blood concentration flow is an indicator.

Other applications may become apparent.

Although the invention has been described with reference to certain specific embodiments, various modifications thereof will be apparent to those skilled in the art without departing from the spirit and scope of the invention as outlined in the claims appended hereto. The entire disclosures of all references recited above are incorporated herein by reference.

We claim:

1. A computer-implemented digital image processing system for training an image processing unit to determine a human emotion being experienced by a human subject, the system comprising:
a computer-readable memory comprising a plurality of sequences of RGB images obtained during a time span for a plurality of human subjects, each of the sequences of RGB images being labelled with one of the plurality of known identifiable human emotions being experienced by the respective human subject, each RGB image comprising a red channel, a green channel and a blue channel, each of the red channel, green channel and blue channel each having a bit length of more than one bit; and
an image processing unit comprising one or more processors in communication with the computer-readable memory, the image processing unit executable to:
generate a set of bitplane images from each of the plurality of sequences of RGB images, each bitplane image being an image formed by isolating a particular bit position within a red, green or blue channel of the corresponding RGB image;
determine a set of high SNR bitplanes, the high SNR bitplanes being a subset of the bitplane images from each of the plurality of sequences of RGB images that optimize hemoglobin concentration differentiation between the identifiable human emotions, by removing effects of at least one of cardiac, respiratory, and blood pressure data from the captured images; and
train a machine learning model utilizable by the image processing unit to determine the human emotion experienced by the human subject by analyzing spatial changes in the hemoglobin concentration obtainable in the set of high SNR bitplanes during the captured image sequences and associating each of the identifiable human emotions with the spatial changes.

2. The system of claim 1, wherein the labels of the plurality of known identifiable human emotions are determined by capturing image sequences from the human subjects being exposed to stimuli known to elicit specific emotional responses.

3. The system of claim 2, wherein the image processing unit is further configured to determine whether each captured image shows a visible facial response to the stimuli.

4. The system of claim 3, wherein each captured image is discarded where the image processing unit determines that there is not a visible facial response.

5. The system of claim 1, wherein removing effects of at least one of cardiac, respiratory, and blood pressure data from the captured images comprises using data from at least one of an EKG machine, a pneumatic respiration machine, and a continuous blood pressure measuring system.

6. The system of claim 1, wherein the image processing unit further performs de-noising.

7. The system of claim 6, wherein the de-noising comprises one or more of Fast Fourier Transform (FFT), notch and band filtering, general linear modeling, and independent component analysis (ICA).

8. The system of claim 1, wherein analyzing the spatial changes in the hemoglobin concentration comprises analyzing spatial changes in the hemoglobin concentration in one or more regions of interest, the one or more regions of interest comprising at least one of forehead, nose, cheeks, mouth, and chin.

9. The system of claim 8, wherein the image processing unit further manipulates bitplane vectors using image subtraction and addition to maximize the signal differences in the regions of interest between different emotional states across the image sequence.

10. The system of claim 9, wherein the subtraction and addition are performed in a pixelwise manner.

11. A computer-implemented method for training an image processing unit to determine a human emotion being experienced by a human subject, the method using a plurality of sequences of RGB images obtained during a time span for a plurality of human subjects, each of the sequences of RGB images being labelled with one of the plurality of known identifiable human emotions being experienced by the respective human subject, each RGB image comprising a red channel, a green channel and a blue channel, each of the red channel, green channel and blue channel each having a bit length of more than one bit, the method comprising:
generating a set of bitplane images from each of the plurality of sequences of RGB images, each bitplane image being an image formed by isolating a particular bit position within a red, green or blue channel of the corresponding RGB image;
determining a set of high SNR bitplanes, the high SNR bitplanes being a subset of the bitplane images from each of the plurality of sequences of RGB images that optimize hemoglobin concentration differentiation between the identifiable human emotions, by removing effects of at least one of cardiac, respiratory, and blood pressure data from the captured images; and
training a machine learning model utilizable by the image processing unit to determine the human emotion experienced by the human subject by analyzing spatial changes in the hemoglobin concentration obtainable in the set of high SNR bitplanes during the captured image sequences and associating each of the identifiable human emotions with the spatial changes.

12. The method of claim 11, wherein the labels of the plurality of known identifiable human emotions are determined by capturing image sequences from the human subjects being exposed to stimuli known to elicit specific emotional responses.

13. The method of claim 12, further comprising determining whether each captured image shows a visible facial response to the stimuli.

14. The method of claim 13, wherein each captured image is discarded it is determined that there is not a visible facial response.

15. The method of claim 11, wherein removing effects of at least one of cardiac, respiratory, and blood pressure data from the captured images comprises using data from at least one of an EKG machine, a pneumatic respiration machine, and a continuous blood pressure measuring system.

16. The method of claim 11, further comprising performing de-noising.

17. The method of claim 16, wherein the de-noising comprises one or more of Fast Fourier Transform (FFT), notch and band filtering, general linear modeling, and independent component analysis (ICA).

18. The method of claim 11, wherein analyzing the spatial changes in the hemoglobin concentration comprises analyzing spatial changes in the hemoglobin concentration in one or more regions of interest, the one or more regions of interest comprising at least one of forehead, nose, cheeks, mouth, and chin.

19. The method of claim 18, further comprising manipulating bitplane vectors using image subtraction and addition to maximize the signal differences in the regions of interest between different emotional states across the image sequence.

20. The method of claim 19, wherein the subtraction and addition are performed in a pixelwise manner.
